(19) United States
(12) Patent Application Publication
(10) Pub. No.: US 2017/0266204 A1
(43) Pub. Date: Sep. 21, 2017

(54) ONAPRISTONE METABOLITE COMPOSITIONS AND METHODS

(71) Applicant: ARNO THERAPEUTICS, INC., Flemington, NJ (US)

(72) Inventors: Stefan Proniuk, Austin, TX (US); Souzan Yanni, Chapel Hill, NC (US)

(21) Appl. No.: 15/464,085

(22) Filed: Mar. 20, 2017

Related U.S. Application Data
(60) Provisional application No. 62/310,944, filed on Mar. 21, 2016.

(51) Int. Cl. A61K 31/575; A61K 45/06

(52) U.S. Cl. CPC A61K 31/575; A61K 45/06

(57) ABSTRACT

Methods and pharmaceutical compositions for inhibiting the activity of the progesterone receptor with methyl onapristone. Aspects include methods of administering methyl onapristone to a patient in an amount sufficient to achieve a blood or tissue concentration of at least about 100 nM.

This application claims priority to U.S. Provisional Patent Application Ser. No. 62/310,944, filed Mar. 21, 2016. The above referenced application is incorporated herein by reference as if restated in full.

All references cited herein, including but not limited to patents and patent applications, are incorporated by reference in their entirety.

### BACKGROUND

Onapristone (ONA) is an anti-progestin drug and progesterone receptor antagonist which was originally developed for contraceptive use. However, it has demonstrated substantial activity in advanced breast cancer. It is thought that ONA binds to the progesterone receptor (PR), preventing the PR from binding to DNA, and thereby inhibiting or eliminating PR-induced transcription. See, e.g., Klijn et al., Progesterone antagonists and progesterone receptor modulation in the treatment of breast cancer, Steroids, v. 65, pp. 825-830 (2000); Jonat et al., The clinical efficacy of progesterone antagonists in breast cancer, Endocrine Therapy of Breast Cancer, pp. 117-124.

Following administration to a patient, drugs are metabolized by the body according to their chemical structure, route of administration, and the genetic and non-genetic factors of the patient. Yanni, Translational ADMET for Drug Therapy: Principles, Methods, and Pharmaceutical Applications. Wiley ISBN-13: 978-1118838273 (2015), pp 63-109. Metabolites produced in the body may be pharmaco-active compounds which exert desirable effects or undesirable side effects. Baillie and Rettie Role of biotransformation in drug-induced toxicity: influence of intra and inter-species differences in drug metabolism. Drug Metab Pharmacokinet (2010); 26:pp 15-29. The undesirable side effects caused by the metabolites (e.g. liver toxicity, autoimmune effects, idiosyncratic interactions, or interaction with other drugs, etc.) vary depending on drug concentration in the systemic circulation, and on how readily the drug is cleared from the body.

Clinical development of onapristone, at elevated doses, was initially terminated due to liver toxicity. Although the mechanism of liver toxicity was not determined, current clinical efforts are directed to reducing side effects of onapristone. What is needed is a compound with the anti-progestin activity of onapristone and a metabolic profile with fewer negative side effects.

### SUMMARY

In one aspect, metabolites of onapristone are provided (e.g., mono-N-desmethyl onapristone). Mono-N-desmethyl onapristone (also referred to herein as methyl onapristone, or N-desmethyl onapri stone, (8S, 11R,13R,14S,17S)-17-hydroxy-17-(3-hydroxypropyl)-13-methyl-11-(4-(methylamino)phenyl)-6,7,8,11,12,13,14,15,16,17-decahydro-1H-cyclopenta[a]phenanthren-3(2H)-one or M1) and has the following structure:

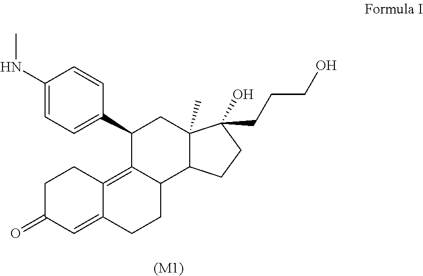

Metabolism

As described herein, twenty seven (27) metabolites are produced (in vivo) in chimeric mice with humanized liver following administration of onapristone while nine metabolites are formed in vitro after oxidative metabolism of onapristone in human liver microsomes fraction (HLM). In contrast as described herein, one metabolite (N-didesmethyl onapristone (M2)) is produced in vitro in HLM following administration of M1.

Aspects described herein provide methods of inhibiting progesterone activity by administering methyl onapristone to cells expressing the progesterone receptor. Further aspects provide methods of inhibiting progesterone activity in a patient having a progesterone receptor positive tumor by administering methyl onapristone to the patient. In another aspect, pharmaceutical compositions comprising methyl onapristone and a pharmaceutically acceptable carrier are provided.

### BRIEF DESCRIPTION OF DRAWINGS

FIG. 1 shows an exemplary LC-MS (liquid chromatography-mass spectrometry) chromatogram of a control sample from chimeric mice;

FIG. 2 shows an exemplary LC-MS chromatogram of samples from chimeric mice after repeated doses of onapristone at 10 mg/kg per day;

FIG. 3 shows an exemplary LC-MS chromatogram of samples from chimeric mice after repeated doses of onapristone at 30 mg/kg/day;

FIG. 4 shows an exemplary elimination pathway in urine for chimeric mice after dosing with onapristone; and

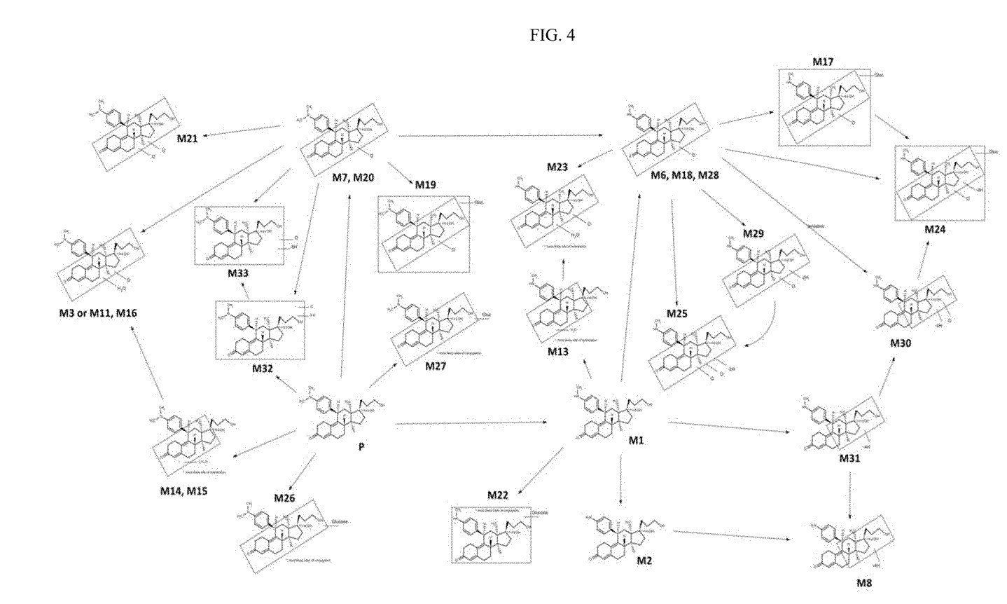

FIG. 5 shows the exemplary results of luciferase assays measuring the effect of methyl onapristone on transcription in 293T (human embryonic kidney cells) and WMPY (a prostatic stromal cell line) cells transfected with PR and PRE-luciferase reporter constructs; and.

### DETAILED DESCRIPTION

Before describing several exemplary aspects described herein, it is to be understood that the invention is not limited to the details of construction or process steps set forth in the following description. The aspects described herein are capable of being practiced or being carried out in various ways.

As described herein, the onapristone metabolite M1 has an improved metabolic profile compared to onapristone. For example, the metabolism of onapristone (e.g., through activity of liver enzyme CYP3A) is about ten times faster than the metabolic rate of M1 (e.g., forming M2 through activity of liver enzymes CYP3A and CYP2C). Therefore, higher doses of onapristone, with the accompanying higher risk of negative side effects, may be necessary to produce the same therapeutic effect provided by M1 at a lower dose.

Onapristone metabolism produces twenty seven (27) metabolites, in contrast to M1 metabolism which produces one oxidative metabolite (M2). Thus, the risk of toxicity from M1 administration is reduced compared to onapristone because fewer metabolites are produced. In addition, M1 pharmacotherapy has reduced risk of drug-drug interaction due to its metabolic profile compared to ONA.

Since the rate of ONA metabolism is ten times higher than the rate of M1 metabolism, ONA may need to be administered to a patient at a ten times higher concentration than M1 to obtain the same systemic exposure. Thus, M1 can be dosed at a higher concentration without increasing the risk of toxicity, resulting in a potentially greater therapeutic effect or at a substantially lower dose to obtain a potentially equal therapeutic effect

Furthermore, as mentioned above, ONA administration results in the production of a number of metabolites, including M1. M1 also binds the progesterone receptor (PR). Consequently, M1 competes for the PR receptor, reducing the effect of ONA on the PR receptor. In contrast, M1 administration will not result in drug competition for binding to the PR receptor.

M1 was previously identified as a metabolite of ONA but its biological activity, metabolic profile, and suitability as a drug was not assessed. Jang and Benet Drug Metabolism disposition 1997; 25: 1119-1122 Cytochrome P450 3A4-mediated N-demethylation of the antiprogestins lilopristone and onapristone.

As described herein, the kinetic analysis indicates that the rate of metabolism of ONA to form M1 is ˜10×-faster than the rate of metabolism of M1 to M2. The metabolism of ONA to M1 is catalyzed by P450 enzyme, CYP3A, while the metabolism of M1 to M2 catalyzed by CYP3A and CYP2C.

In one aspect, ONA can be involved in auto drug-drug interaction (DDI). For example, ONA may inhibit its own metabolism and M1 metabolism, as indicated from clinical and in vitro data. Therefore, accumulation of ONA and M1 in the blood circulation may cause unwanted DDI side effects. In contrast, administration of M1 may avoid such unwanted side effects because M1 metabolism produces one metabolite (M2).

The rate of metabolism of onapristone (Vmax 1 and Vmax2) to form N-mono desmethyl onapristone is 10 times the rate of metabolism of N-monodesmethyl onapristone to N-di demethylated onapristone as shown in the Table 1 below:

TABLE 1
Rate of Metabolism (Vmax 1 and Vmax 2) of Onapristone
to Form M1 and Metabolism of M1 to Form M2 in
Female and Male Human Liver Microsomes

| | Kₘ 1 | Kₘ 2 | Vₘₐₓ 1 | Vₘₐₓ 2 |
|---|---|---|---|---|
| Metabolism | (μM) | (μM) | (pmol/min/mg) | (pmol/min/mg) |
| | Female | | | |
| ONA to M1 | 7.80 | 139 | 1517 | 8715 |
| M1 to M2 | 0.75 | 175 | 106 | 738 |
| | Male | | | |
| ONA to M1 | 7.80 | 99 | 1292 | 7213 |
| M1 to M2 | 0.96 | 129 | 72 | 748 |

As shown in Table 1, the Vmax for ONA metabolism in females is 1517 and the Vmax in females for M1 metabolism is 106 pmol/min/mg microsomal protein. The Vmax for ONA metabolism in males is 1292 pmol/min/mg microsomal protein and the Vmax in males for M1 metabolism is 72 pmol/min/mg microsomal protein. Thus, in both females and males, it requires about 10 times higher dose of onapristone than M1 to achieve systemic exposure and the same efficacy. Consequently, the safety profile of M1 is likely to be higher with fewer side effects than onapristone. In this aspect, M1 could be provided to patients in a higher dose, and presumably higher efficacy than onapristone but with reduced side effects.

Aspects described herein provide methods of inhibiting the activity of the progesterone receptor in cells of a patient that express the progesterone receptor by administering methyl onapristone to the patient and exposing the cells to methyl onapristone.

In another aspect, the cells are selected from breast, blood, prostate, brain, meningiomas, prostate, ovarian, endometrial, uterine leiomyoma, lung, bile duct, lung, bone, esophagus, kidney, pancreas, intestine, stomach, urinary tract, skin, liver, thyroid, and uterine cells.

Further aspects provide methods of inhibiting progesterone activity in a patient with a progesterone receptor positive tumor by administering methyl onapristone to the patient. In one aspect, the amount of methyl onapristone administered to the patient is from about 1 to about 10 mg. In yet another aspect, the amount of methyl onapristone administered to the patient is sufficient to achieve a blood or tissue concentration of at least about 100 nM. In another aspect, M1 can be used as a contraceptive.

In another aspect, the number of metabolites produced in response to treatment with methyl onapristone is no more than about four.

Further aspects provide methods of administering an anti-tumor compound (e.g., everolimus, trastuzumab, TM1-D, anti-HER2 drugs, bevacizumab, paclitaxel, docetaxel, taxanes, doxorubicin, liposomal doxorubicin, pegylated liposomal doxorubicin, anthracyclines, anthracenediones, abiraterone, enzulutamide, carboplatin, cisplatin, 5-FU, gemcitabine and cyclophosphamide) to the patient receiving methyl onapristone.

Aspects described herein provide pharmaceutical compositions comprising methyl onapristone and a pharmaceutically acceptable excipient, carrier, or diluent (e.g., hydroxyproyl methyl cellulose, microcrystalline cellulose, lactose or mannitol). The pharmaceutical composition can be provided, for example, in a unit dosage form (e.g., tablets, pills, capsules, and troches. In another aspect, the methyl onapristone is present in an amount from about 2 to about 200 mg or from about 1 to about 10 mg.

In yet another aspect, an additional active pharmaceutical agent (e.g., antitumor agent, a hormone, a steroid, or a retinoid) can be provided. Additional active ingredients can include, for example, everolimus, trastuzumab, TM1-D, anti-HER2 drugs, bevacizumab, paclitaxel, docetaxel, taxanes, doxorubicin, liposomal doxorubicin, pegylated liposomal doxorubicin, anthracyclines, anthracenediones, abiraterone, enzulutamide, carboplatin, cisplatin, 5-FU, gemcitabine and cyclophosphamide.

Metabolite Profile of Onapristone

Summary

The metabolite profile of onapristone was determined in a chimeric mouse with humanized liver model following oral doses of onapristone at 10 and 30 mg/kg/day. Metabolites were identified using liquid chromatography-mass spectrometry (LC-MS) and mass spectroscopy on pooled plasma, urine, and liver samples. Twenty-seven metabolites were identified in urine samples across dose and sampling time (e.g., 0-24 hours of dosing for each time point).

Twenty-one of the metabolites resulted from phase I reactions (e.g., oxidations, N-demethylation dehydrogentation, and combination of these pathways). Six metabolites resulted from phase II reactions.

LC-MS profile identifies four metabolites generated from M1 by phase I oxidation reaction (M2, M31, M8) and one by phase II glucuronidation reaction (M22). These metabolites were detected in urine samples after onapristone administration for 32 days at target doses of 10 and 30 mg/kg/day.

Reference Standards

Onapristone (AR-18) and Mono-methyl onapristone (AR-19) were used as reference standards:

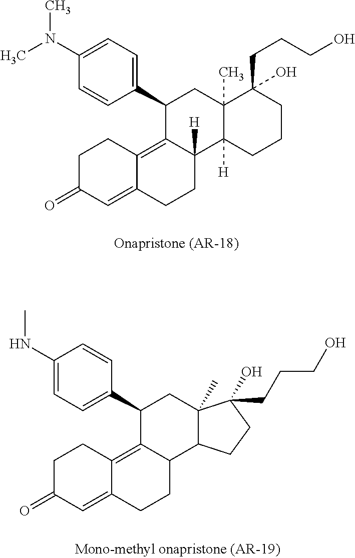

Methods

Use of Human Liver Microsomes to Study Oxidative Metabolism of Onapristone

Human liver microsomes (HLM) have been frequently used to study the oxidative metabolism of drugs in vitro. Mohutsky M, Wrighton S, Ring B. In vitro metabolism: subcellular fractions. In: Pearson P, Wienkers L, editors. Handbook of drug metabolism. New York: Informa Healthcare; 2009. pp. 445-464. As described herein, onapristone (e.g., at a concentration of 5μM incubated in presence of NADPH-regenerating system as cofactor) was incubated with a 0.5 mg/mL human liver microsomes pool from 50 donors, purchased from BD-Genetest. The incubation was conducted at various time points 0-60 min at 37° C. At the end of incubation, the reactions were quenched, processed, and analyzed by LC-MS/MS.

Chimeric Mouse Model for Determining Metabolic Profile

Mice with humanized livers were generated as described by Meuleman et al., Hepatology 2005, 41 (4):847-856. This humanized liver model is routinely used to assess the metabolism and toxicities of drugs. Kamimura H, Nakada N, Suzuki K, et al. Assessment of chimeric mice with humanized liver as a tool for predicting circulating human metabolites. Drug Metab Pharmacokinet 2010;25(3):223-235. Two weeks following birth, mice were transplanted with primary hepatocytes isolated from the liver of a 27-year old female Caucasian donor (purchased from BD Genetest). All animals were housed and kept under standard animal protocols. Animals also were observed during the experiment for signs of illness, abnormal behavior and appearance, and any sign of toxicity, or mortality. Animals were treated orally by gavage with 0, 10, and 30 mg/kg of drug once a day up to 31 days. At the end of treatments, blood samples, urine, and liver were collected and processed for LC-MS/MS metabolite identification and profiling.

Biological Samples

Non-acidified urine, plasma, and liver samples were obtained after multiple oral administrations of onapristone in chimeric mice at the target doses of 10 and 30 mg/kg/day as described below.

Experimental Design

LC-MS/MS was performed on samples pooled from animals treated at the same dose for each time point as shown below for six plasma samples, six urine samples, and two liver samples. Herein, metabolites in urine samples are shown.

TABLE 2
Summary of Metabolites Detected by LC-MS In Pooled
Urine Samples From Chimeric Mice After Repeated Dosing
With Onapristone

| | Urine | | | | | |
|---|---|---|---|---|---|---|
| Metabolite | 10 mg/kg/day | | | 30 mg/kg/day | | |
| ID | D1 | D11 | D32 | D1 | D11 | D32/D28 |
| Parent (P) | — | — | — | — | Ls | |
| M1 | X | X | X | X | X | X |
| M2 | X | X | X | X | X | X |
| M3 or M11 or | ls | X | X | X | X | X |
| isomer | | | | | | |
| M6 or isomer | X | ls | X | X | X | X |
| M7 | — | ls | ls | X | X | X |
| M8 | X | X | X | X | X | X |
| M13 | X | X | X | X | X | X |
| M14 | X | X | X | X | X | X |
| M15 | X | X | X | X | X | X |
| M16 | — | ls | ls | ls | X | X |
| M17 | ls | ls | ls | ls | X | X |
| M19 | ls | ls | ls | X | X | X |
| M20 | ls | ls | ls | X | X | X |
| M21 | — | — | — | X | X | X |
| M22 | ls | X | ls | X | ls | X |
| M23 | X | X | X | X | X | X |
| M24 | ls | ls | ls | X | X | X |
| M25 | ls | — | ls | X | X | X |
| M26 | — | — | — | ls | ls | X |
| M27 | — | — | — | X | X | X |
| M28 | — | ls | ls | X | X | X |
| M29 | — | ls | ls | X | X | X |
| M30 | X | X | X | X | X | X |
| M31 | X | X | X | X | X | X |
| M32 | ls | X | X | X | X | X |
| M33 | — | — | — | ls | X | X |

X = metabolite detected by LC-MS;
— = metabolite not detected;
ls = low LC-MS signal (intensity ≦150).
Time point: D 1 = samples collected in day 1 of dosing;
D11—samples collected in day 11 of dosing;
D32—samples collected in day 32 of dosing;
D32/28 = samples pooled in day 32 and day 28.

LC-MS Chromatograms

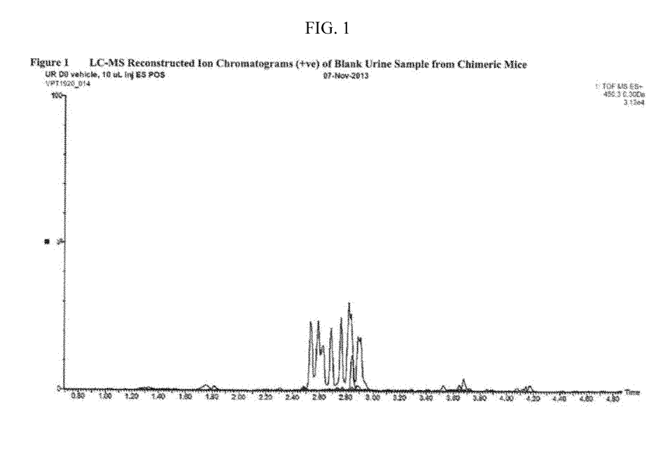

FIG. 1 is an LC-MS reconstructed ion chromatogram of a blank urine sample from chimeric mice receiving vehicle. No significant peaks are shown.

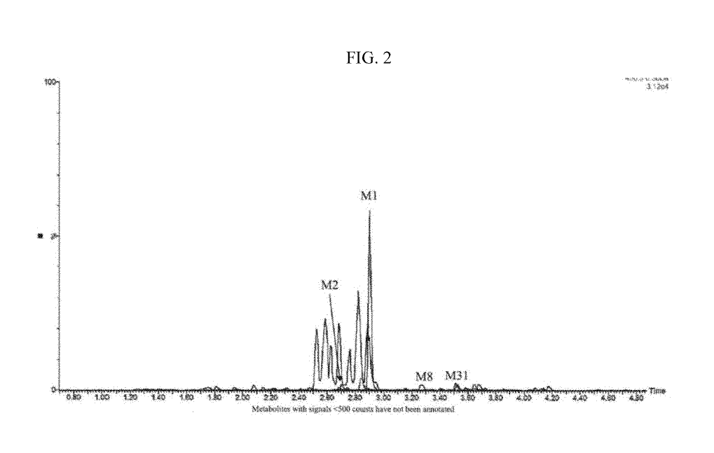

FIG. 2 is an LC-MS reconstructed ion chromatogram of a day 32 pooled urine sample from chimeric mice after repeated doses of onapristone at 10 mg/kg/day. Significant peaks for metabolites M1 and M2 are shown. Less significant peaks for metabolites M8 and M31 are also shown.

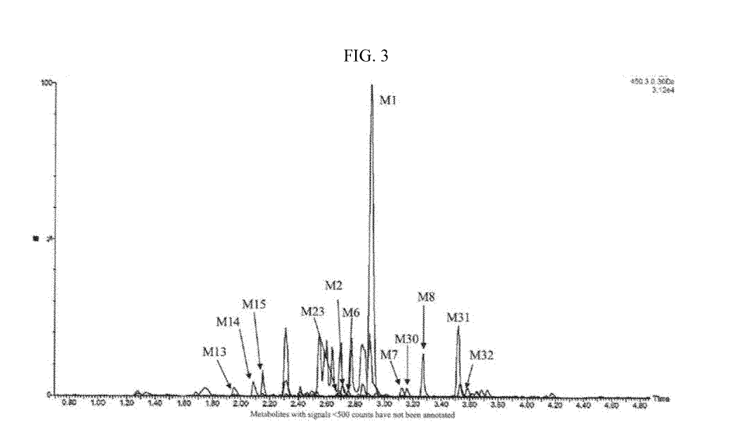

FIG. 3 is an LC-MS reconstructed ion chromatogram of day 28/32 pooled urine samples from chimeric mice after repeated doses of onapristone at 30 mg/kg/day. Peaks for metabolites M13, M14, M15, M23, M2, M6, Ml, M7, M30, M8, M31, and M32 are shown.

Methyl Onapristone Activity

The inhibitory activity of methyl onapristone positive cells was demonstrated in 293T and WMPY (prostatic stromal) luciferase reporter cell lines.

Luciferase assays on AR-19 (methyl-onapristone)

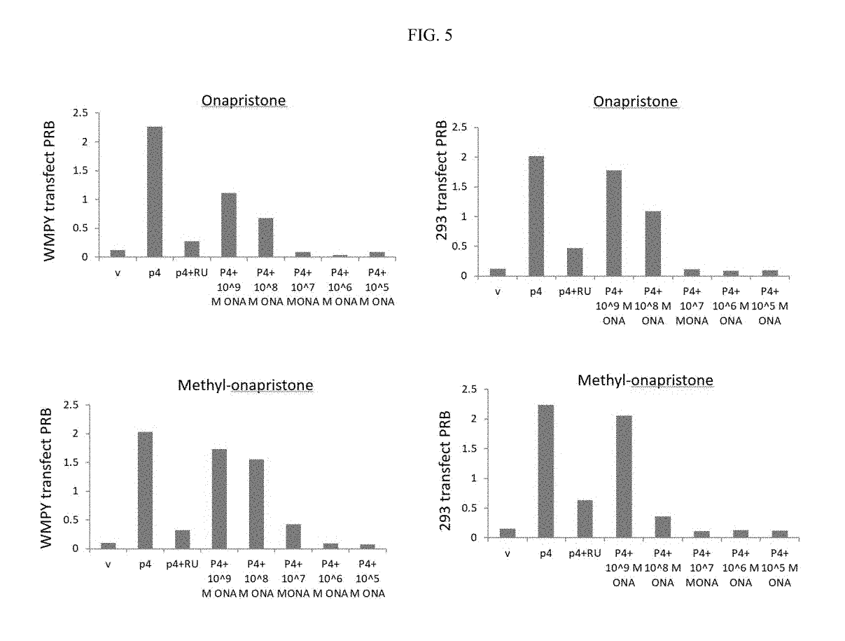

293T and WMPY cells were transfected with progesterone receptor (PR) and PRE-luciferase reporter. Cells were then treated with P4, P4+RU486 or P4+ increasing doses of onapristone or methyl onapristone (AR-19). Experiments were repeated in triplicate. As shown in FIG. 5, onapristone and methyl onapristone have similar effects with respect to inhibiting PR transcriptional activity. In one aspect, 100 nM of onapristone and M1 can fully inhibit PR transcriptional activity. In this aspect, a concentration of 100 nM of onapristone (and 10 nM of M1) was achieved in blood circulation after dosing onapristone at 10 mg QD. Because of the metabolic stability of M1 compared to ONA, a blood circulation of 100 nM M1 can be achieved after dosing M1 at 1 mg.

Methyl onapristone and pharmaceutical compositions comprising methyl onapristone can be used to treat a patient in need of treatment as described herein. The terms “treat,” “prevent,” or similar terms, as used herein, do not necessarily mean 100% or complete treatment or prevention. Rather, these terms refer to various degrees of treatment or prevention of a particular disease (e.g., 100%, 90%, 80%, 70%, 60%, 50%, 40%, 30%, 20%, 10%, 5%, or 1%) as recognized in the art as being beneficial.

The terms “treatment” or “prevention” also refer to delaying onset of a disease for a period of time or delaying onset indefinitely.

The term “treatment” or “treating” refers to administering a drug or treatment to a patient or prescribing a drug to a patient or a third party (e.g., caretaker, family member, or health care professional) administers the drug or treatment.

Methyl onapristone and pharmaceutical compositions comprising methyl onapristone also encompass hydrates or solvates of ONA polymorphic or crystalline forms (e.g., hemihydrate, monohydrate, dihydrate, trihydrate and the like). Hydrates or solvates of ONA may be prepared by contacting ONA with water or a solvent under suitable conditions to produce the hydrate or solvate of choice.

Any of the methyl onapristone and pharmaceutical compositions comprising methyl onapristone described herein can be administered or used as starting materials to be administered orally, parenterally (IV, IM, depot-1M, SQ, and depot-SQ), sublingually, intranasally (inhalation), intrathecally, topically, or rectally. Dosage forms known to those of skill in the art are suitable for delivery of methyl onapristone and pharmaceutical compositions comprising methyl onapristone described herein.

Methyl onapristone and pharmaceutical compositions comprising methyl onapristone can be formulated into suitable pharmaceutical preparations such as tablets, capsules, or elixirs for oral administration or in sterile solutions or suspensions for parenteral administration. Methyl onapristone and pharmaceutical compositions comprising methyl onapristone can be formulated into pharmaceutical compositions using techniques and procedures well known in the art.

In one aspect, about 2 to about 200 mg of methyl onapristone and pharmaceutical compositions comprising methyl onapristone, or a physiologically acceptable salt, pro-drug, or co-crystal thereof can be compounded or used as a starting material for compounding with a physiologically acceptable vehicle, carrier, excipient, binder, preservative, stabilizer, flavor, etc., in a unit dosage form as called for by accepted pharmaceutical practice. The amount of active substance in compositions or preparations comprising methyl onapristone and pharmaceutical compositions comprising methyl onapristone is such that a suitable dosage in the range indicated is obtained.

In another aspect, the compositions can be formulated in a unit dosage form, each dosage containing from about 1 mg to about 1200 mg, 2.5 mg to about 200 mg, and 1 to 10 mg of the active ingredient. The term “unit dosage form” refers to physically discrete units suitable as unitary dosages for human subjects and other mammals, each unit containing a predetermined quantity of active material calculated to produce the desired therapeutic effect, in association with one or more suitable pharmaceutical excipients.

In one aspect, one or more of the methyl onapristone and pharmaceutical compositions comprising methyl onapristone are mixed with or used as starting materials mixed with a suitable pharmaceutically acceptable carrier to form compositions. Upon mixing or addition of the compound(s), the resulting mixture may be a solution, suspension, emulsion, or the like. Liposomal suspensions may also be used as pharmaceutically acceptable carriers. These may be prepared according to methods known to those skilled in the art. The form of the resulting mixture depends upon a number of factors, including the intended mode of administration and the solubility of the compound in the selected carrier or vehicle. In one aspect, the effective concentration is sufficient for lessening or ameliorating at least one symptom of the disease, disorder, or condition treated and may be empirically determined.

Pharmaceutical carriers or vehicles suitable for administration of the methyl onapristone and pharmaceutical compositions comprising methyl onapristone described herein include any such carriers suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

In another aspect, if the methyl onapristone and pharmaceutical compositions comprising methyl onapristone exhibit insufficient solubility, methods for solubilizing may be used. Such methods are known and include, but are not limited to, using co-solvents such as dimethylsulfoxide (DMSO), using surfactants such as TWEEN, and dissolution in aqueous sodium bicarbonate. Derivatives of the compounds, such as salts or prodrugs, may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

In another aspect, the methyl onapristone and pharmaceutical compositions comprising methyl onapristone described herein may be prepared with carriers that protect them against rapid elimination from the body, such as time-release formulations or coatings. Such carriers include controlled release formulations, such as, but not limited to, microencapsulated delivery systems. The active compound can be included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

In another aspect, the methyl onapristone and pharmaceutical compositions comprising methyl onapristone described herein can be enclosed in multiple or single dose containers. The enclosed compounds and compositions can be provided in kits, for example, including component parts that can be assembled for use. For example, a methyl onapristone polymorphic compound can be used as a starting material for a lyophilized form and a suitable diluent may be provided as a separated component for combination prior to use. A kit may include methyl onapristone and a second therapeutic agent for co-administration. The methyl onapristone compound and second therapeutic agent may be provided as separate component parts. A kit may include a plurality of containers, each container holding one or more unit dose of the methyl onapristone compound described herein. In one aspect, the containers can be adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of the methyl onapristone compound in the pharmaceutical composition will depend on dissolution, absorption, metabolism, and excretion rates of the active compound, the dosage schedule, and amount administered as well as other factors known to those of skill in the art.

In another aspect, the active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound can be provided in a composition that protects it from the acidic environment of the stomach or any potential precipitation. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a glidant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action. The ONA polymorphic compounds can be used, for example, in combination with an antitumor agent, a hormone, a steroid, or a retinoid. The antitumor agent may be one of numerous chemotherapy agents (e.g., everolimus, trastuzumab, TM1-D, anti-HER2 drugs, bevacizumab, paclitaxel, docetaxel, taxanes, doxorubicin, liposomal doxorubicin, pegylated liposomal doxorubicin, anthracyclines, anthracenediones, carboplatin, cisplatin, 5-FU, gemcitabine and cyclophosphamide). In one aspect, the antitumor agent does not exhibit drug-drug interactions with respect to methyl onapristone.

In one aspect, solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerin, propylene glycol, or other synthetic solvent; antimicrobial agents such as benzyl alcohol and methyl parabens; antioxidants such as ascorbic acid and sodium bisulfate; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include, but are not limited to, physiological saline, phosphate buffered saline (PBS), and solutions containing thickening and solubilizing agents such as glucose, polyethylene glycol, polypropyleneglycol, and mixtures thereof. Liposomal suspensions including tissue targeted liposomes may also be suitable as pharmaceutically acceptable carriers. These may be prepared according to methods known in the art.

In another aspect, the methyl onapristone compounds may be prepared with carriers that protect the compound against rapid elimination from the body, such as timerelease formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, hydroxyl propyl methyl cellulose (HPMC), other cellulose derivatives, and the like. Methods for preparation of such formulations are known to those skilled in the art.

In yet another aspect, compounds employed in the methods of the disclosure may be administered enterally or parenterally. When administered orally, compounds employed in the methods of the disclosure can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, they can be of the sustained release type so that the compounds employed in the methods described herein need to be administered only once or twice daily.

The oral dosage forms can be administered to the patient once, twice, three or four times daily. The methyl onapristone compounds described herein can be administered either three or fewer times, or even once or twice daily. Hence, the methyl onapristone employed in the methods of the disclosure be administered in oral dosage form. Whatever oral dosage form is used, they can be designed so as to protect the compounds employed in the methods described herein from the acidic environment of the stomach. Enteric coated tablets are well known to those skilled in the art. In addition, capsules filled with small spheres each coated to protect from the acidic stomach, are also well known to those skilled in the art.

The terms “therapeutically effective amount” and “therapeutically effective period of time” are used to denote treatments at dosages and for periods of time effective to reduce neoplastic cell growth. As noted above, such administration can be parenteral, oral, sublingual, transdermal, topical, intranasal, or intrarectal. In one aspect, when administered systemically, the therapeutic composition can be administered at a sufficient dosage to attain a blood level of the compounds from about 0.01 μM to about 20 μM. For localized administration, much lower concentrations than this can be effective, and much higher concentrations may be tolerated. One of skill in the art will appreciate that such therapeutic effect resulting in a lower effective concentration of the ONA polymorphic compound may vary considerably depending on the tissue, organ, or the particular animal or patient to be treated. It is also understood that while a patient may be started at one dose, that dose may be varied overtime as the patient's condition changes. In one aspect, the ONA polymorphic compounds can be used to inhibit the growth of tumors derived from tissue including, but not limited to, breast, brain, meningiomas, prostate, ovarian, endometrial, uterine leiomyoma, lung, and uterine tissues.

It should be apparent to one skilled in the art that the exact dosage and frequency of administration will depend on the particular compounds employed in the methods of the disclosure administered, the particular condition being treated, the severity of the condition being treated, the age, weight, general physical condition of the particular patient, and other medication the individual may be taking as is well known to administering physicians who are skilled in this art.

Although the above description refers to particular aspects, it is to be understood that these aspects are merely illustrative. It will be apparent to those skilled in the art that various modifications and variations can be made to the polymorphic forms and methods described herein. Thus, it is intended that the present description include modifications and variations that are within the scope of the appended claims and their equivalents.

What is claimed is:

1. A method of inhibiting the activity of the progesterone receptor in cells of a patient that express the progesterone receptor comprising, administering methyl onapristone to the patient and exposing the cells to methyl onapristone.

2. The method of claim 1, where in the cells are selected from breast, blood, prostate, brain, meningiomas, prostate, ovarian, endometrial, uterine leiomyoma, lung, bile duct, lung, bone, esophagus, kidney, pancreas, intestine, stomach, urinary tract, skin, liver, thyroid, and uterine cells.

3. A method of inhibiting progesterone activity in a patient with a progesterone receptor positive tumor, comprising administering methyl onapristone to the patient.

4. The method of claim 3, wherein the amount of methyl onapristone administered to the patient is from about 1 to about 10 mg.

5. The method of claim 3, wherein the amount of methyl onapristone administered to the patient is sufficient to achieve a blood or tissue concentration of at least about 100 nM.

6. The method of claim 3, wherein the number of metabolites produced in response to treatment with methyl onapristone is no more than about four.

7. The method of claim 1, wherein methyl onapristone has a contraceptive effect in the patient.

8. The method of claim 3, further comprising administering an anti-tumor compound selected from the group consisting of everolimus, trastuzumab, TM1-D, anti-HER2 drugs, bevacizumab, paclitaxel, docetaxel, taxanes, doxorubicin, liposomal doxorubicin, pegylated liposomal doxorubicin, anthracyclines, anthracenediones, abiraterone, enzulutamide, carboplatin, cisplatin, 5-FU, gemcitabine and cyclophosphamide to the patient.

9. A pharmaceutical composition comprising methyl onapristone and a pharmaceutically acceptable excipient, carrier, or diluent.

10. The pharmaceutical composition of claim 9, in a unit dosage form.

11. The pharmaceutical composition of claim 10, wherein the unit dosage form is selected from the group consisting of tablets, pills, capsules, and troches.

12. The pharmaceutical composition of claim 9, where the methyl onapristone is present in an amount from about 2 to about 200 mg.

13. The pharmaceutical composition of claim 12, where the methyl onapristone is present in an amount from about 1 to about 10 mg.

14. The pharmaceutical composition of claim 9, further comprising at least one additional active pharmaceutical agent.

15. The pharmaceutical composition of claim 14, wherein the additional active pharmaceutical agent is selected from the group consisting of an antitumor agent, a hormone, a steroid, or a retinoid.

16. The pharmaceutical composition of claim 14, wherein the additional active ingredient is selected from the group consisting of everolimus, trastuzumab, TM1-D, anti-HER2 drugs, bevacizumab, paclitaxel, docetaxel, taxanes, doxorubicin, liposomal doxorubicin, pegylated liposomal doxorubicin, anthracyclines, anthracenediones, abiraterone, enzulutamide, carboplatin, cisplatin, 5-FU, gemcitabine and cyclophosphamide.
